(19) United States
(12) Patent Application Publication
(10) Pub. No.: US 2012/0083002 A1
(43) Pub. Date: Apr. 5, 2012

(54) Methods For Modulating The Development Of Dopamine Neuron By The Dopamine D2 Receptor And Compositions Thereof

(75) Inventors: Ja hyun BAIK, Seoul (KR); Sung yul KIM, Seoul (KR)

(73) Assignee: Korea University Industry And Academy Cooperation Foundation, Seoul (KR)

(21) Appl. No.: 13/271,987

(22) Filed: Oct. 12, 2011

Related U.S. Application Data
(62) Division of application No. 11/995,743, filed on Jan. 15, 2008, now abandoned.

(30) Foreign Application Priority Data
Apr. 4, 2006 (KR) 10-2006-0030700

(51) Int. Cl. G01N 33/566

(52) U.S. Cl. 435/7.21

(57) ABSTRACT

The present invention relates to a composition for modulating the activation of Nurr1, the composition comprising an agonist or an antagonist of a dopamine D2 receptor, methods for modulating the activation of Nurr1 by the dopamine D2 receptor, a method and composition for treating Nurr1-related diseases using the dopamine D2 receptor, and methods for screening a modulator of a dopamine D2 receptor of a test compound. Accordingly, the activation of Nurr1 can be modulated by treating the dopaminergic neurons with the agonist or the antagonist of the dopamine D2 receptor, thereby enhancing or inhibiting generation of the dopaminergic neurons.

### TECHNICAL FIELD

The present invention relates to a composition for modulating the activation of Nurr1, the composition comprising an agonist or an antagonist of a dopamine D2 receptor, methods for modulating the activation of Nurr1 by the dopamine D2 receptor, a method and composition for treating Nurr1-related diseases using the dopamine D2 receptor, and methods for screening a modulator of a dopamine D2 receptor of a test compound.

### BACKGROUND ART

Since dopamine was discovered in the 1950s, its functions in the brain, has been intensively researched. Dopamine is a neurotransmitter and dopamine-producing cells are generated within the embryonic ventral mesencephalon, and this process has been shown to require a complex network consisting of numerous transcription factors and signaling pathways (Perrone-Capano et al., Neurosci Biobehav Rev., 2000 Jan; 24(1):119-24; Simon H H et al., Ann N Y Acad Sci, 2003 June; 991: 36-47; Riddle R and Pollock J D, Brain Res Dev Brain Res., 2003 Dec. 30; 147(1-2):3-21.). With regard to functions of dopamine, it has been known that dopamine is essentially associated with brain functions in a variety of ways, including motion function, cognitive function, sensory function, emotional function, and autonomous function (e.g., regulation of appetite, body temperature, or sleep). Therefore, the dopaminergic modulation is useful in the treatment of extensive disorders adversely affecting brain functions. In practice, psychiatric and neurodegenerative disorders are treated by drugs using interaction between the dopamine system and receptor in the brain.

Dopamine receptors can be categorized into several types (e.g., D1, D2, D3, D4, D5, and so on). It is known that these dopamine receptors involve different functions in certain areas of the brain, and many studies are being attempted as to possible treatments for related disorders using compounds capable of specifically binding these receptors. For example, WO 99/09025 discloses certain 2-(4-aryl or heteroaryl-piperazin-1-ylmethyl-1H-indole derivatives, which interact with dopamine D4 receptor. Further, WO 1996/02249 discloses thiadiazole compounds useful as dopamine D3 receptor ligands. WO 1995/33729 describes that novel compounds including 4-phenylpiperazine, 4-phenyl-piperadine and 4-phenyl-1,2,3,6-tetrahydropiridine compound effectively act on central serotonergic receptors, e.g. 5-HT1A, and dopamine D2 receptors.

Meanwhile, Nurr1, which is a transcription factor belonging to steroid thyroid hormone receptors (Law, et al., Mol. Endocrinol., 1992, 6:2129) and expressed in dopaminergic cells (Zetterstrom, et al., Mol. Brain Res., 1996, 41:111), is considered to serve in development of dopaminergic neurons in the mesencephalon. In this regard, in order to investigate the role of Nurr1, Nurr1 null mutant mice were generated. The Nurr1 null mutant mice failed to generate mesencephalon dopaminergic neurons, and died soon after birth, suggesting that Nurr1 played a key role in induction of mesencephalon dopaminergic neurons(Zetterstrom, et al., Science, 1997, 276:248-250; Saucedo-Cardenas, et al., Proc. Natl. Acad. Sci. USA, 1998, 95:4013-18; Castillo, et al., Mol. Cell Neurosci., 1998, 11:36-46). However, many factors networking inherent to these signalling mechanisms associated with the development of dopaminergic neurons have yet to be clearly elucidated.

In this context, in the course of exploring the role of dopamine specifically binding to dopamine D2 receptor, the inventors of the present invention completed the present invention based on these findings that Nurr1 and Ptx3 (paired-like homeodomain transcription factor 3) activation levels in neurons are different relative to the presence or absence of dopamine D2 receptors, which interact with Nurr1 by activation of extracellular signal regulated kinase (ERK).

### DISCLOSURE OF THE INVENTION

To solve the above problems, it is an object of the present invention to provide compositions for modulating the activation of Nurr1 and the development of dopaminergic neurons, the compositions comprising an agonist or antagonist of a dopamine D2 receptor.

It is another object of the present invention to provide methods for modulating the activation of Nurr1 and the development of dopaminergic neurons by treatment with an agonist or antagonist of a dopamine D2 receptor.

It is still another object of the present invention to provide a method for screening modulators of a dopamine D2 receptor, the method comprising contacting a test compound with dopaminergic neurons, and measuring an increased or decreased activation level of Nurr1.

It is a further object of the present invention to provide methods and compositions for treating Nurr1-related diseases by treatment with an agonist or antagonist of a dopamine D2 receptor.

### BRIEF DESCRIPTION OF THE DRAWINGS

FIG. 1 shows TH-positive cells in mesencephalic cultures from wild-type (WT) mice and D2R−/− E14 mice lacking dopamine D2 receptor and the numbers of TH-positive neurons after treatment with 1-methyl-4-phenylpyridinium (MPP⁺). FIG. 1A is a representative photomicrograph of wild-type control (CT), D2R−/− control, WT treated with 10 μM MPP⁺ for 24 hours, and D2R−/− treated with 10 μM MPP⁺ for 24 hour (Scale bar, 50 μm), FIG. 1B shows proportion in the change of number of TH-positive neurons from mesencephalic cultures from WT (n=5) and D2R−/− (n=7) embryonic mice after treatment with 1-10 μM MPP⁺, and FIG. 1C shows percentage of number of TH-positive neurons from mesencephalic cultures from WT (N=5) and D2R−/−(N=7) embryonic mice after treatment with 1-10 μM MPP⁺.

FIG. 2 shows stereological analysis of number of TH-positive neurons in WT and D₂R−/− mice, in which FIG. 2A shows representative coronal sections of mice of embryonic day 14 (E14), postnatal day 30 (P30) and postnatal day (P60) stages with TH-positive neurons in substantia nigra (SN) and ventral tegmental area (VTA) visualized by immunohistochemistry (Scale bars: higher magnification, 400 μm; lower magnification, 200 μm), and FIG. 2B shows counted data of TH-positive neurons in the mesencephalon of E14 stage mice and in the SN or VTA of P30 and P60 stage mice.

FIG. 3 shows stereological analysis of number of Nurr1-positive neurons in WT and D2R−/− mice, in which FIG. 3A shows representative coronal sections of mice of E14, P30 and P60 stages with Nurr1-positive neurons in substantia nigra (SN) and ventral tegmental area (VTA) visualized by immunohistochemistry (Scale bars: higher magnification, 400 μm; lower magnification, 200 μm), and FIG. 3B shows counted data of TH-positive neurons in the mesencephalon of E14 stage mice and in the SN or VTA of P30 and P60 stage mice.

FIG. 4 shows developmental expression of Ptx3 mRNA in WT and D2R−/− mice, as confirmed by developmental stages (ages) of E15, P1, 1M, 2M, 4M, 6M (aged 1, 2, 4, 6 months), and 1Y (aged 1 year), in which FIG. 4A shows results of reverse transcription (RT)-PCR analysis for Ptx3 transcripts conducted from the midbrains of WT and D2R−/− mice, and FIG. 4B shows data plotted in percentages for Ptx3 mRNA levels, respectively, in relation to mRNA levels of β-actin gene used as an internal standard.

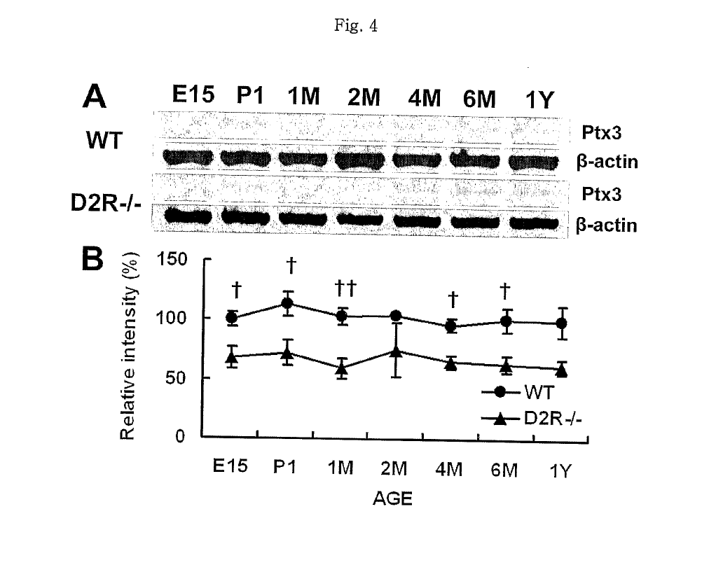

FIG. 5 illustrates the role of MAPK (MAP kinase) related signaling mechanism in NurRE-dependent transcriptional activation of the luciferase reporter gene after D2R stimulation in HEK293T cells, in which FIG. 5A shows luciferase activity (%) relative to the concentration of dopamine in HEK293T cells transiently transfected with either a combination of D2R and Nurr1 or Nurr1/D2R alone, and FIG. 5B shows luciferase activity (%) relative to the concentration of dopamine with or without treatment of a D2R antagonist haloperidol (1 μM), FIG. 5C shows luciferase activity (%) relative to the concentration of dopamine with or without treatment of a Gαi inhibitor pertussis toxin (PTX) (100 ng/ml, 12 hours), FIG. 5D shows luciferase activity (%) relative to the concentration of dopamine with or without treatment of an MEK inhibitor PD98059 (10 μM, 30 minutes), FIG. 5E shows the effect of RasN17, which is a mutant form of Ras, on the NurRE-dependent transcriptional activation of the luciferase reporter gene after D2R stimulation, FIG. 5F shows the effect of a PKA inhibitor H-89 (1 μM, 20 minutes) on the NurRE-dependent transcriptional activation of the luciferase reporter gene after D2R stimulation, and FIG. 5G shows comparison results of experiments for relative luciferase activity (%), conducted with D2R and dopamine D1 receptor (D1R) and a D1R specific derivative SKF81297.

FIG. 6 shows the effect of D2R activation in the number of TH neurons and the enhancement of morphological changes in mesencephalic neuronal cultures from WT and D2R−/− mice, in which FIG. 6A is a representative diagram illustrating treatment with quinpirole (Q), haloperidol plus quinpirole (H+Q), PD98059 (PD), and PD98059 plus quinpirole (PD+Q), with a control group (CT) on the mesencephalic neuronal cultures from WT and D2R−/− mice (scale bar, 100 μm), FIG. 6B shows the quantitative analysis of the numbers of TH-positive neurons in mesencephalic neuronal cultures from WT and D2R−/− mice, and FIG. 6C shows the qualitative analysis of the average length of the neurites of TH-positive neurons in mesencephalic neuronal cultures from WT and D2R−/− mice.

FIG. 7 shows MAP kinase activation induced by D2R stimulation in mesencephalic dopaminergic neurons from WT and D2R−/− mice, in which FIG. 7A shows representative immunofluorescence images of phosphorylated ERK (p-ERK) by quinpirole (Q) (10 μM, 15 minutes) in TH-positive neurons from WT and D2R−/− mice, and FIGS. 7B and 7C show the effect of treatment with a control group (CT), quinpirole (Q), haloperidol plus quinpirole (H+Q), PD98059 (PD), and PD98059 plus quinpirole (PD+Q), as analyzed by Western blot and quantitative relative intensity analysis, on the colocalization of p-ERK and TH-positive neurons from WT and D2R−/− mice mesencephalic neuronal cells.

FIG. 8 shows Nurr1 activation induced by D2R stimulation in mesencephalic dopaminergic neurons from WT and D2R−/− mice, from which mesencephalic cultures were then treated with quinpirole (Q) for 6 hours and fixed to then immunostained with anti-TH antibody and anti-Nurr1 antibody, in which FIG. 8A shows representative immunofluorescence images of Nurr1 positive cells among TH-positive neurons, activated by quinpirole, and FIG. 8B shows the result of quantitative analysis of a ratio of Nurr1 positive cells to TH-positive neurons.

### BEST MODE FOR CARRYING OUT THE INVENTION

In an aspect, the present invention is directed to compositions for modulating the activation of Nurr1 comprising an agonist or antagonist of a dopamine D2 receptor, and the development of dopaminergic neurons.

The term “dopamine D2 receptor” used in the present invention means a binding site to which dopamine, etc. released from the dopaminergic neurons binds. When dopamine binds to the dopamine D2 receptor, the stimulation of dopamine D2 receptor can elicit the activation of Nurr1 and the development of dopaminergic neurons.

The term “development” used in the present invention means differentiation or proliferation of dopaminergic neurons.

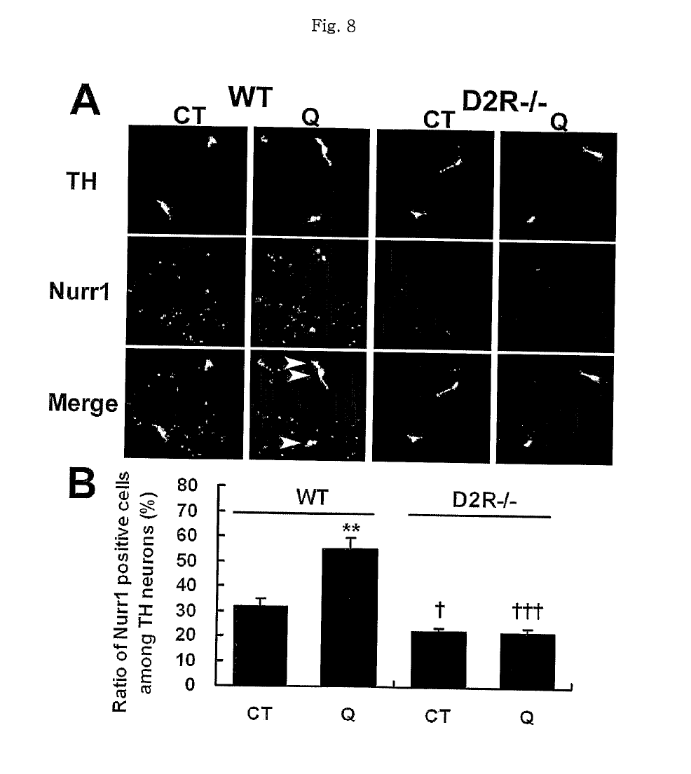

The term “agonist” used in the present invention means an agent binding to a dopamine D2 receptor, enhancing Nurr1 activation. Specifically, a dopamine D2 receptor agonist according to an embodiment of the present invention may comprise sumanirole, quinpirole, cabergoline, bromocriptine, and so on. In a specific embodiment, when WT and D2R−/− mice were treated with quinpirole, only neurons from the WT mice exhibited enhanced Nurr1 activation (FIG. 8).

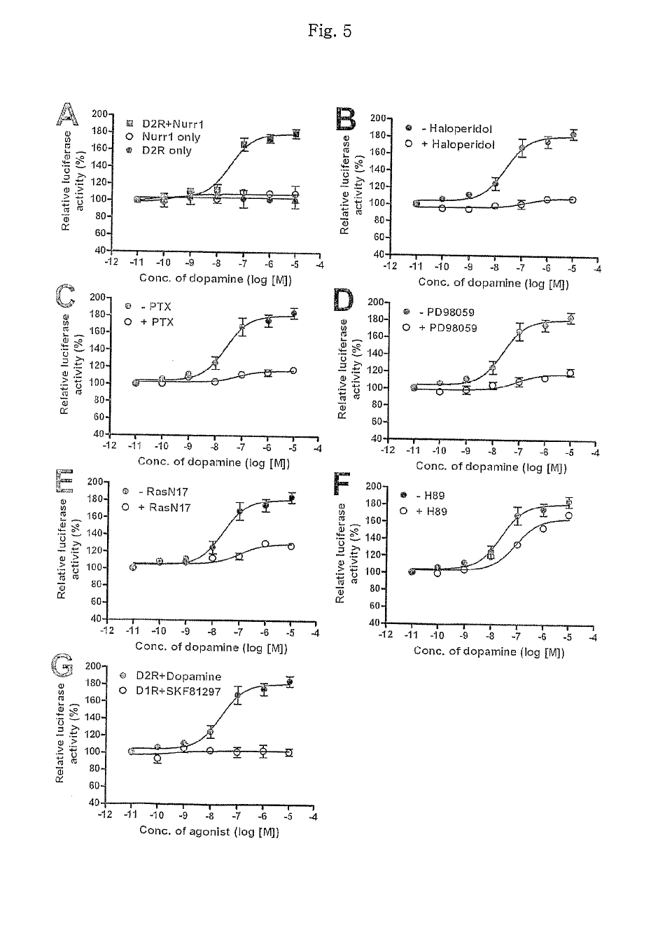

The term “antagonist” used in the present invention means an agent binding to a dopamine D2 receptor, decreasing or inhibiting Nurr1 activation. Specifically, in an embodiment, a dopamine D2 receptor antagonist according to an embodiment of the present invention may comprise haloperidol, spiperone, remoxipride, and so on. In a specific embodiment, when WT and D2R−/− mice were treated with haloperidol, Nurr1 activity levels were reduced, compared with cases when WT and D2R−/− mice were not treated with haloperidol (FIG. 5B).

The term “modulating or modulated activation of Nurr1” used in the present invention means to increase or decrease the relative Nurr1 activity depending on the concentration of dopamine, or means that the relative Nurr1activity is increased or decreased depending on the concentration of dopamine. The development of dopaminergic neurons can be modulated by regulating Nurr1 activation, and diseases related with Nurr1 activation or dopaminergic neurons can be treated and/or prevented accordingly.

In another aspect, the present invention provides methods for modulating the activation of Nurr1 and the development of dopaminergic neurons by treatment with an agonist or antagonist of a dopamine D2 receptor.

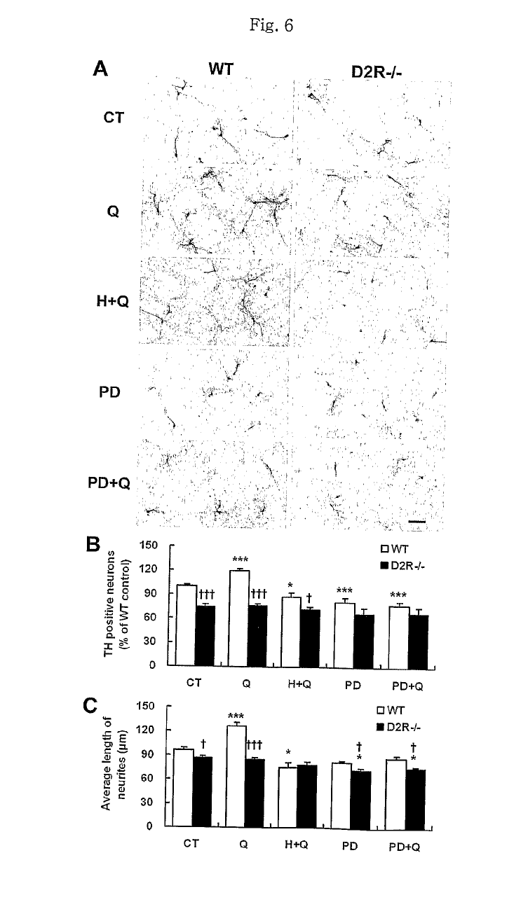

ERK activation is increased or decreased by an agonist or antagonist binding to the dopamine D2 receptor according to the present invention, thereby modulating the development of the dopaminergic neurons. In a specific embodiment, when WT mice were treated with a dopamine D2 receptor agonist quinpirole, the number of dopaminergic neurons and the average length of the neuritis were both increased. By contrast, when D2R−/− mice were treated with quinpirole, there were little changes in the number of dopaminergic neurons and the average length of the neurites. Meanwhile, as shown in FIGS. 6B and 6C, when WT and D2R−/− mice were treated with a dopamine D2 receptor antagonist haloperidol, the number of dopaminergic neurons and the average length of the neurites were decreased or showed insignificant changes.

In another aspect, the present invention provides a method for screening modulators of a dopamine D2 receptor, the method comprising contacting a test compound with dopaminergic neurons, and measuring an increased or decreased activation level of Nurr1.

In still another aspect, the present invention provides a method for screening modulators of a dopamine D2 receptor, the method comprising contacting a test compound with dopaminergic neurons, and measuring an increased or decreased development level of dopaminergic neurons.

The term “test compound” used in the present invention means a compound or drug binding to a dopamine D2 receptor to test whether to enhance or decrease generation of dopamine neurons or to determine the activation level for treatment of Nurr1 related diseases.

The screening method of the present invention comprises contacting the test compound with dopaminergic neurons, and measuring an increased or decreased activation level of Nurr1 or measuring an increased or decreased development level of dopaminergic neurons after the lapse of a predetermined time, which may be carried out in vivo, in situ, and in vitro.

Any methods of determining Nurr1 activation known in the art may be used, for example, western blotting in which quantities of proteins in cells and activation by phosphorylation were indirectly visualized, immunofluorescence staining in which expression levels of proteins in cells were directly visualized, comparison of changes of proteins migrating between cytoplasm and karyoplasm to determine activation levels of transcription factors migrating from the cytoplasm to the karyoplasm, gel retardation assay in which activation of nucleic transcription factors are measured, indirect activation measurement using luciferase-dependent reporter, and so on. In a specific embodiment of the present invention, Nurr1 activation was determined by a Nur-reactive factor (NurRE)-dependent reporter gene activation test method.

Any methods of determining the development level of dopaminergic neurons in the art may be used. For example, differentiation levels of dopaminergic neurons can be determined by neurite outgrowth, increase in the number of neurites, neural migration, or marker protein or mRNA testing according to differentiation level or step. Proliferation levels of dopaminergic neurons can be determined by directly staining and counting the number of dopaminergic neurons, incorporation of radioactive [³H]-thymidine into dopaminergic neurons, incorporation of fluorescent BrdU into dopaminergic neurons, MTT dye reduction, and so on.

In a specific embodiment of the present invention, dopaminergic neurons were specifically stained by immunofluorescence staining and the number of neurons was then measured and the average length and number of neurites were also measured to determine the development level of dopaminergic neurons.

The screening method according to the present invention may further comprise, after measuring the increased or decreased activation level of Nurr1, comparing the measured activation level of Nurr1 with that in the absence of a test compound. If the activation level of Nurr1 in the presence of the test compound was higher than that in the absence of the test compound, the test compound is determined as a potential agonist of the dopamine D2 receptor. On the contrary, if the activation level of Nurr1 in the presence of the test compound was lower than that in the absence of the test compound, the test compound is determined as a potential antagonist of the dopamine D2 receptor.

The screening method according to the present invention may further comprise, after measuring the increased or decreased development level of dopaminergic neurons, comparing the measured development level of dopaminergic neurons with that in the absence of a test compound. If the development level of dopaminergic neurons in the presence of the test compound is higher than that in the absence of the test compound, the test compound is determined as a potential agonist of the dopamine D2 receptor. On the contrary, if the development level of dopaminergic neurons in the presence of the test compound was lower than that in the absence of the test compound, the test compound is determined as a potential antagonist of the dopamine D2 receptor.

In a further aspect, the present invention provides methods and compositions for treating Nurr1-related diseases by treatment with an agonist or antagonist of a dopamine D2 receptor.

The term “treatment” used in the present invention means both therapeutic treatment and preventative measures. Those in need of treatment include those already with neurological disorder or neurological disease as well as those in which the neurodegenerative disorder or neurological disease is to be prevented. While the method of the present invention is not limited to the listed examples, it can be used in treating any mammal requiring therapeutic treatments or measures, including humans, primates, livestock, or animals for breeding, companion or racing, for example, dogs, horses, cats, sheep, pigs, cows, or the like.

The term “Nurr1-related disease” used in the present invention means a disease that may be caused by modulated Nurr1 activity by an agonist or antagonist a dopamine D2 receptor. The Nurr1-related disease may include dopaminergic neuron related diseases (developmental disorder of dopaminergic neurons and impairment in neuroplasticity of dopaminergic neurons). More concretely, the Nurr1-related disease may include neurodegenerative diseases such as Parkinson's syndrome, drug addiction, neuropsychiatric diseases such as depression or post-traumatic stress disorder.

The therapeutic composition of the present invention can be formulated for injection, oral, topical, nasal administration by inhalation or insufflation (either through the mouth or the nose) or buccal, parenteral or rectal administration. The therapeutic composition according to the present invention may also comprise diluents, preservatives, solubilizers, emulsifying agents, adjuvants, excipients and/or carriers. In addition, the therapeutic composition of the present invention may also additives including various buffers (e.g., Tris-HCl, acetate salt, or phosphate salt), pH and ion strength diluents; detergents and disintegrants (e.g., Tween 80, or polysorbate 80), antioxidants (e.g., ascorbic acid, or sodium metabisulfite), preservatives (e.g., thimerosal, or benyl alcohol), and bulking agents (e.g., lactose, or mannitol).

The therapeutic composition of the present invention may be prepared in the form of purified multi-microcapsules provided in granules or pellets. Formulations of the invention suitable for capsule administration may be in the form powder, softly compressed plugs or tablets.

The therapeutic composition of the present invention may include dyes and flavoring agents. For example, proteins (or derivatives) can be formulated (by, for example, a liposome or microsphere capsulation method) and may further be contained in edible products such as cold drinkables comprising dyes and flavoring agents.

The disintegrant may be included in the formulation of a therapeutic as a dry product. Examples of materials which can be used as the disintegrant include, but not limited to, commercially available starch based disintegrants, such as corn starch or potato starch. Some examples of materials which can serve as the disintegrant may also include sodium starch glycolate, amberlite, sodium carboxymethyl cellulose, ultramylopectin, sodium alginate, gelatin, orange peel waxes, acid carboxymethyl cellulose, natural sponges and bentonites. Other type of disintegrant is an insoluble anion exchange resin. Powdered gums may be used as disintegrants and as binders and these can include powdered gums such as agar, Karaya or tragacanth. Alginic acid and its sodium salt are also useful as disintegrants.

An anti-frictional agent may be included in the formulation of the therapeutic to prevent sticking during the formulation process. Lubricants may be used as a layer between the therapeutic and the die wall, and these can include but are not limited to; stearic acid including its magnesium and calcium salts, polytetrafluoroethylene (PTFE), liquid paraffin, vegetable oils and waxes. Soluble lubricants may also be used such as sodium lauryl sulfate, magnesium lauryl sulfate, polyethylene glycol of various molecular weights, Carbowax 4000 or 6000.

Glidants that might improve the flow properties of the drug during formulation and to aid rearrangement during compression might be added. The glidants may include starch, talc, pyrogenic silica and hydrated silicoaluminate.

To aid dissolution of the therapeutic into the aqueous environment a surfactant might be added as a wetting agent. Surfactants may include anionic detergents such as sodium lauryl sulfate, dioctyl sodium sulfosuccinate and dioctyl sodium sulfonate. Cationic detergents might be used and could include benzalkonium chloride or benzethomium chloride. The list of potential non-ionic detergents that could be included in the formulation as surfactants are lauromacrogol 400, polyoxyl 40 stearate, polyoxyethylene hydrogenated castor oil 10, 50 and 60, glycerol monostearate, polysorbate 40, 60, 65 and 80, sucrose fatty acid ester, methyl cellulose and carboxymethyl cellulose.

Liquid formulations suitable for oral administration include products in forms of solutions, syrups, suspensions, dry products for constitution having water or other suitable vehicles for use.

Meanwhile, the liquid formulations may include pharmaceutically acceptable suppositories, which are exemplified by suspensions such as sorbitol, syrup, cellulose derivatives or edible hydrogenated lipid), emulsifying agents, such as lecithin or acacia, non-aqueous vehicles such as almond oil, oily esters, ethyl alcohol or fractionated vegetable oil, and preservatives such as methyl- or propyl-p-hydroxybenzoates or sorbic acid. Such preparations may also include buffering agents, salts, dyes, flavoring agents, sweetening agents, and so on.

The therapeutic composition of the present invention can also be delivered nasally. Nasal delivery allows the passage of a pharmaceutical composition of the present invention to the blood stream directly after, administering the therapeutic product to the nose, without the necessity for deposition of the product in the lung. Formulations for nasal delivery include those with dextran or cyclodextran.

For inhalation administration, the pharmaceutical composition of the present invention may be conventionally delivered in the form of an aerosol spray presentation or spray gun from pressurized packs with the use of a suitable propellant, e.g. dichlorodifluoromethane, dichlorotetrafluoroethane, carbon dioxide or other suitable gas.

The pharmaceutical composition of the present invention may be formulated or parenteral administration by injection e.g. by bolus injection or continuous infusion. Formulations for injection may be presented in unit dosage form e.g. in ampoules or in multi-dose containers, with an added preservative. The pharmaceutical composition of the present invention may take such forms as suspensions, solutions or emulsions in oily or aqueous vehicles, and may contain formulatory agents such as surfactants, stabilizers and/or dispersing agents. Alternatively, the active ingredient of the pharmaceutical composition may be in powder form for constitution with a suitable vehicle, e.g. sterile pyrogen-free water, before use.

The therapeutic composition of the present invention may also be formulated in rectal compositions such as suppositories or retention enemas, e.g. containing conventional suppository bases such as cocoa butter or other glycerides.

The therapeutic composition of the present invention may be administered parenterally or topically, for example, by transmucosal administration, e.g., oral, nasal, or rectal administration, or by transdermal administration. For example, preferred administration may include, but not limited to, parenteral administration such as intravenous injection, intramuscular, transdermal, subcutaneous, intraperitoneal, intraventricular, and intracranial administration.

The therapeutic composition of the present invention is administered in a pharmaceutically effective amount. In the present invention, the term “pharmaceutically acceptable effective amount” is used to mean an amount enough for applications having a reasonable benefit-risk ratio to treat or prevent diseases. The effective dosage level is selected in accordance with a variety of factors including the type and severity of disease; the age, weight, sex, and medical condition of a patient; patient's sensitivity to particular drugs; the time of administration, the route of administration and the rate of release; the treatment period; and factors including drugs in combination with or together with the composition of the present invention and other factors well known in the medical field. In general, the pharmaceutically acceptable amount of the present invention ranges from between 0.01 mg per kg of body weight per day (mg/kg/day) to about 500 mg/kg/day.

The present invention may be better understood by reference to the following non-limiting Examples. The following examples are presented in order to more fully illustrate the preferred embodiments of the invention. The present invention is not restricted to the following Examples, and it is understood by one skilled in the art that many variations are possible within the spirit and scope of the present invention.

### EXAMPLE 1

### Preparation of Animals and Primary Culture of Mesencephalic Neuronal Cells

D2R−/− mice and wild-type (WT) mice were obtained by mating D2R−/− mice and heterozygous mice, purchased from Institut de Genetiqul et Biologie Moleculaire et celluaire (Strasbourg, France), and their genotypes were identified by Southern hybridization analyses (An J J et al., Mol Cell Neurosci. 2004, 25: 732-741). Insemination was confirmed by vaginal plug and considered to be embryonic day 0 (E0). Pregnant mothers were killed at E14 in accordance with Society for Neuroscience Guidelines. To prepare primary mesencephalic neuronal cultures, the mesencephalon dissected from 14 day gestation mouse embryo was incubated with 0.1% of trypsin in HBSS for 10 minutes at 37° C. and triturated with a constricted Pasteur pipette in high-glucose DMEM media supplemented with 10% FBS (Invitrogen, San Diego, Calif.), 1.4 mM L-glutamine, and 6.0 g/L glucose. The neurons were plated at 1.0×10⁵ cells per 18×18 mm coverslip (Marienfeld, Lauda-Konigshofen, Germany) or 2.0×10⁵ cells per six-well plates precoated with 50 μg/ml poly-D-lysine and 2 μg/ml laminin (Sigma, St. Louis, Mo.). The neurons were maintained at 37° C. in a humidified 5% CO₂ atmosphere in Neurobasal media supplemented with B27 and GlutaMax-1.

### EXAMPLE 2

### Effect of Absence of D2R on Number of Neurons

To determine whether the absence of dopamine D2 receptor (D2R) might affect the number of dopaminergic neurons, dopaminergic neurons, which were isolated from mesencephalons of wild-type (WT) and D2R−/− embryonic mice, were incubated on slides with 1.0×10⁵ cells and precoated with 50 μg/ml poly-D-lysine and 2 μg/ml laminin (Sigma, St. Louis, Mo.) at 37° C. for 5 days, followed by performing immunofluorescence staining. The immunofluorescence staining was performed such that primarily cultured dopaminergic neurons were fixed with 4% paraformaldehyde for 20 minutes at room temperature (RT) and blocked for 1 hour in PBS containing 5% normal horse serum and 0.2% Triton X-100. Then, the neurons were incubated with a rabbit polyclonal anti-tyrosine hydroxylase (TH) (1:1000; Pel-Freez, Rogers, Ariz.) in PBS containing 1% normal horse serum and 0.2% Triton X-100 at 4° C. for over 16 hours, and followed by staining according to avidin-biotin immunohistochemical procedures (Vector Laboratories, Burlingame, Calif.). Using a microscope equipped with an metamorph imaging system (Universal Imaging Corporation, West Chester, Pa.) in 20 randomly selected fields per each slide, cell counts and morphometric analysis were made in randomly selected unbiased counting frames (>40 frames out of 81 grids were counted).

To determine whether the absence of D2R might affect the number of dopaminergic neurons, 1-Methyl-4-phenylpyridinium (MPP⁺) (Research Biochemical, Inc) was added to the primary culture medium and cell counting analysis was made. In detail, dopaminergic neurons, which were isolated from mesencephalons of WT and D2R−/− embryonic mice, were inoculated with 1.0×10⁵ cells on slides precoated with 50 μg/ml poly-D-lysine and 2 μg/ml laminin (Sigma, St. Louis, Mo.) and incubated in Neurobasal media supplemented with B27 and GlutaMax-1 for 4 days. An MMP⁺ stock was prepared by dissolving in fresh culture media for neuronal cultures, and at 5 day in vitro, the neurons were replaced with fresh culture media without B27 supplement, followed by adding the MMP⁺ stock at concentrations ranging from 1 to 10 μM for 24 hours for incubating. Immunofluorescence staining per slide was performed using polyclonal anti-tyrosine hydroxylase (TH) and cell counting was made.

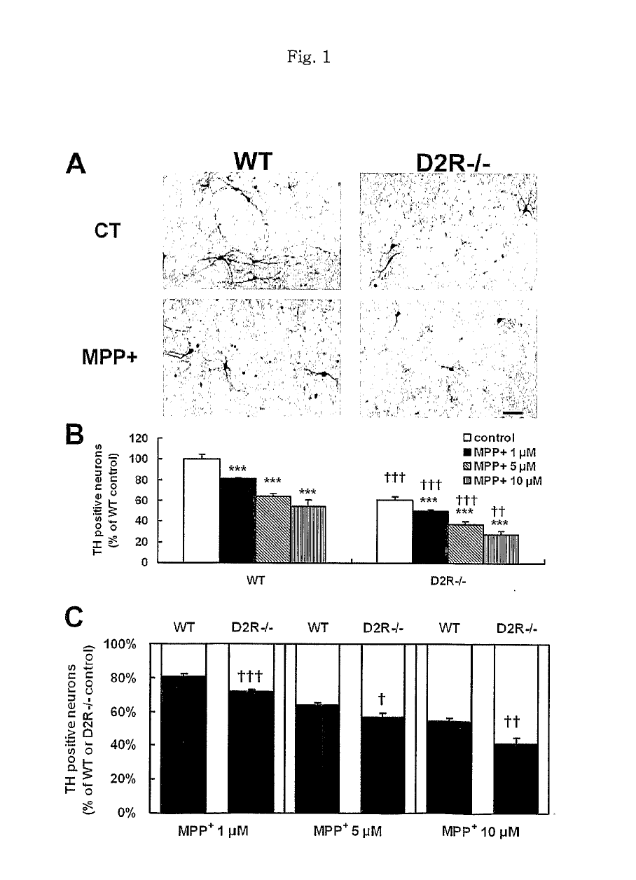

The cell counting analysis showed that treatment with MPP⁺ to the primary mesencephalic dopaminergic neuronal cultures resulted in a more significant loss of dopaminergic neurons in the D2R−/− mice than in WT mice (FIGS. 1B and 1C). Particularly, at a concentration of 10 μM, the number of surviving TH-positive neurons in the D2R−/− mice was only 40%, which is a significant loss compared to the number of primary mesencephalic dopaminergic neuronal cultures in the WT mice, i.e., 54% (FIG. 1C).

### EXAMPLE 3

### Effect of Absence of D2R on Development of Dopaminergic Neurons

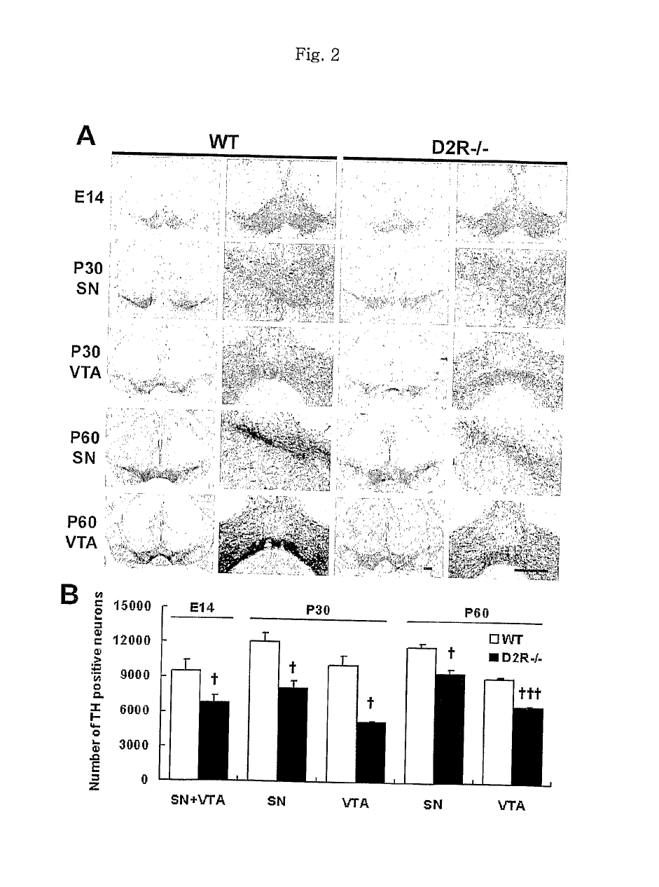

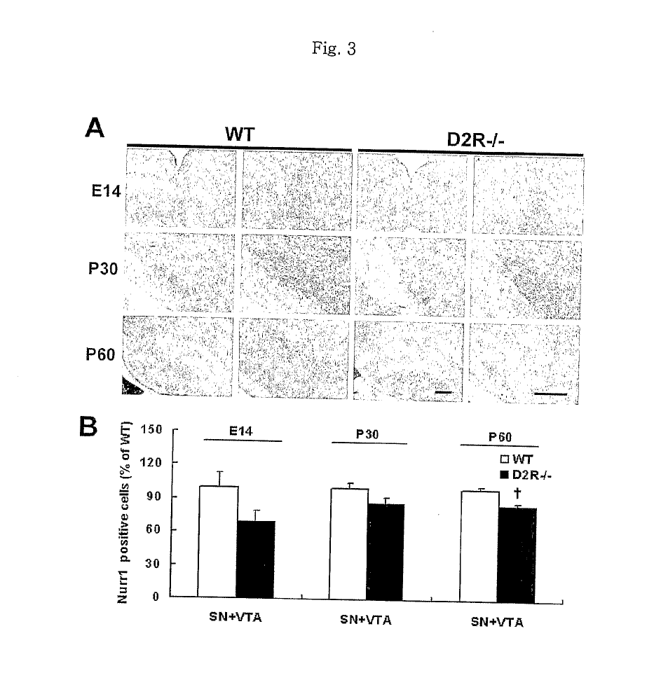

To determine whether the absence of dopamine D2 receptor (D2R) might affect the development of D2R, sections were prepared from WT and D2R−/− mice at E14 and P30 stages, respectively. TH-positive cells in substantial nigra (SN) and ventral tegmental area (VTA) in are counted and a transcription factor Nurr1 known to be expressed uniquely in the SN and VTA was stained to determine expression levels of TH and Nurr1 (FIGS. 2 and 3). In detail, heads of WT and D2R−/− mice were fixed in 4% paraformaldehyde and soaked in an OCT solution. Then, free-floating cryostat sections (40 μm) were serially prepared and treated with anti-TH antibody and anti-Nurr1 body, followed by immunohistochemistry. The immunohistochemistry was performed such that the sections were treated with a mouse polyclonal anti-TH (1:1000; Pel-freez, Rogers, Ariz.) or rabbit polyclonal anti-Nurr1 (M-196, 1:200; Santa Cruz Biotechnology, Santa Cruz Calif.), followed by staining according to avidin-biotin immunohistochemical procedures (Vector Laboratories, Burlingame, Calif.).

The result indicated that the number of TH-positive neurons in the VTN had decreased in D2R−/− mice in E14 stage, to 70% of the levels measured in the WT mice (FIGS. 2A and 2B). Also, in P30 and P60 stages, the number of TH-positive neurons in the SN and VTN had decreased in the D2R−/− mice to about 60% of the levels measured in the WT mice (FIGS. 2A and 2B). The number of Nurr1-positive cells expressed in midbrains of D2R−/− mice in the embryonic stage was reduced to 70% of the levels measured in the WT mice. In P30 and P60 stages, the number of Nurr1-positive cells in the D2R−/− mice was still reduced, showing 85% of the number of Nurr1-positive cells of WT mice (FIG. 3).

### EXAMPLE 4

### Change in the Expression of Ptx3 in the absence of D2R

To determine whether or not the absence of D2R affects the expression of Ptx3 specific to the development of dopaminergic neurons, expression levels of Ptx3 in WT and D2R null mice were compared. Total RNA was prepared from isolated mesencephalon of mice brain using LiCl RNA extraction buffer. First-strand cDNAs were generated from total RNA using reverse transcription with random primer by denaturing at 90° C. for 4 minutes, annealing at room temperature for 10 minutes, and extending at 42° C. for 50 minutes. The following primers were used to amplify target cDNA: PTX3, 5′-AGGACGGCTCTCTGAAGAA-3′, 5′-TTGACCGAGTTGAAGGCGAA-3′; β-actin, 5′-GATG ACGATATCGCTGCGCT-3′ and 5′-GCTCATTGCCGATAGTGATGACCT-3′. Conditions for PCR amplifications were as follows: 94° C. for 5 minutes, 30 cycles at 94° C. for 1 minute, 60° C. for 1 minute, 72° C. for 1 minute, and final extension at 72° C. for 7 minutes. The PCR products were run on 1.5% agarose gels containing EtBr (ethidium bromide) (0.5 μg/ml), to mark and visualize the PCR products using a gel documentation system 2000 (Bio-Rad, Hercules, Calif.).

### EXAMPLE 5

### D2R-Mediated Nurr1 Activation

To investigate the effect of D2R on the number of TH-positive neurons and Nurr1 expression, Nur response element (NurRE)-dependent reporter gene activation assay was carried out to determine whether or not D2R activation might induce Nurr1 activation (Philips et al, Mol Cell Biol., 1997, 17:5946-5951; Maira et al, Mol Cell Biol., 1999, 19:7549-7557). HEK293T cells distributed from Korean cell line Bank were cultured in DMEM (Dulbeco's modified eagle's medium; Invitrogen, Carlsbad, Calif.) supplemented with 10% FBS (fetal bovine serum; Invitrogen, Carlsbad, Calif.) and transfected the same with dopamine receptors, Nurr1, and NurRE gene using a jetPEI transfection reagent (Qbiogene, Carlsbad, Calif.). In detail, 5˜7×10⁵ cells confluent monolayers of HEK293 cells were transfected with 1.5 μg of pSV-D2R or pSV-D₂R, 1.5 μg of pCMX-Nurr1, 1.5 μg of pXP1-luc containing POMC gene promoter and NurRE (pXP1-NurRE-luc), and 0.5 μg of pCH110. In case of the transfection with Ras dominant-negative mutant, cells were transfected with 1.0 pg of pMT-RasN17 or pSK-null vector, 1.0 μg of pSV-D2R, 1.0 μg of pCMX-Nurr1, 1.0 μg of pXP1-NurRE-luc, and 0.5 μg of pCH110. After 3 hours, the transfection mixture was replaced with fresh growth medium. Assays were performed 48 hours after transfection. Cells were preincubated overnight in serum-free growth medium before treatment with agonists. The cells were treated with various concentrations of dopamine or SKF38393, respectively, for 6 hours at 37° C. with or without preincubation of haloperidol (1 μM for 5 minutes), pertussis toxin (PTX) (100 ng/ml for 12 hours), H-89 (1 μM for 20 minutes), and PD98059 (10 μM for 30 minutes). After treatment, the cells were lysed and assayed for luciferase activity using the luciferase assay system (Promega, Madison, Wis.), and luminescence was measured using a 96-well luminometer (Microlumat; EG & Berthold, Bad Wilbad, Germany).

The data obtained were fitted to a sigmoid curve with a variable slope factor using nonlinear squares regression in GraphPad Prism software, which is shown in FIG. 5. Referring to FIG. 5A, after transfecting HEK293T cells with D2R, Nurr1, and NurRE-luc, treatment with dopamine at various concentrations showed about 180% of activation measured in the control group, while no change in the activation was observed in the absence of Nurr1 or D2R.

To confirm the specificity to D2R, an experiment for treatment with a D2R antagonist haloperidol was carried out. The experimental result indicated that D2R-mediated Nurr1 activation was inhibited or decreased (FIG. 5B). To confirm D2R-mediated Gαi activation, an experiment for treatment with a Gαi inhibitor PTX was carried out. The experimental result indicated that D2R-mediated PTX activation(FIG. 5C). To confirm whether or not the Nurr1 activation mediated by dopamine receptors might be inhibited by a D2R-mediated MEK inhibitor PD98059, experiments for treatment with or without MEK inhibitor PD98059 were carried out. The experimental results indicated that Nurr1 activation was noticeably reduced in the presence of PD98059 (FIG. 5D).

The expression of the dominant-negative mutant form of Ras, RasN17, resulted in a significant reduction of luciferase activity compared to the control group (FIG. 5E). Experiments were carried out to investigate the effect of a PKA inhibitor H-89 (1 μM for 20 minutes) on transcription activation of NurRE-dependant luciferase reporter gene induced by a dopamine D2 receptor (D2R). The experimental results indicated that the PKA inhibitor did not give rise to a significant change specifically in luciferase activation (FIG. 5F), suggesting that PKA inhibitor had little effect on Nurr1 activation. In this regard, to confirm whether dopamine D1 receptor (D1R) activation might induce Nurr1 activation, an experiment was carried out by treatment with a D1R-specific agonist SKF81297. The experimental result still indicated no significant change in Nurr1 activation (FIG. 5G).

### EXAMPLE 6

### Effect of D2R Agonist on Development of Dopaminergic Neurons

To determine whether D2R-induced ERK signaling and Nurr1 activation could be correlated with the role of D2R in dopaminergic neuronal development, Primary mesencephalic cultures from WT and D2R−/− mice were prepared in the same manner as in EXAMPLE 1 and treated with 1 μM quinpirole as a D2R agonist every 12 hours for 4 days. Thereafter, the treated cells were fixed and stained in the same manner as in EXAMPLE 2 for counting the number TH-positive cells and analyzing the neuritic shape (FIG. 6A). Quinpirole treatment increased the number of TH neurons by 25% in the mesencephalic primary cultures from WT mice, while little increase in the number of TH neurons was not detected in the cultures from D2R−/− mice (FIG. 6B). In addition, quinpirole treatment induced considerable improvement in the neuritic extension of the dopaminergic neurons, as indicated by average length of neurites, in the mesencephalic primary cultures from WT mice, while little change was detected in the cultures from D2R−/− mice (FIG. 6C).

To confirm whether or not the above observed effects of quinpirol occurred specifically via D2R, the mesencephalic neurons were treated with 1 μM haloperidol as a D2R antagonist for 5 minutes. Thereafter, the neurons were treated with 1 μM quinpirol. As a results, haloperidol induced no changes with regard to both the number of TH-positive neurons (FIG. 5B) and neuritic extention (FIG. 5C) in the D2R−/− mice compared to the case in the control groups without haloperidol treatment.

To confirm whether or not D2R-mediated enhancement of dopaminergic neuronal development involves the ERK pathway, the effect of quinpirole was observed by treating the mesencephalic neurons with MEK (MAP kinase) inhibitor PD98059. According to the observation results, PD98059 treatment slightly reduced the number of TH-positive neurons and also reduced the average length of neuritis, compared to the case in the control groups without PD98059 treatment (FIGS. 6B and 6C).

To confirm whether or not quinpirole treatment might induce the activation of ERK and Nurr1 in the dopaminergic mesencephalic neurons, the neurons were stained by immunostaining, and ERK-positive or Nurr1-positive neurons among the immunostained TH-positive neurons were then examined. At the same time, quinpirole-induced activation of ERK was also detected via Western blot analysis. Mesencephalic neurons from WT and D2R−/− mice were primarily cultured in the same manners in EXAMPLES 1 and 2 and treated with 10 μM quinpirole for 15 minutes. Then, cells were fixed in the same manner as in EXAMPLE 2, a rabbit polyclonal anti-TH (1:1000; Pel-Freez) and a mouse monoclonal anti-phosphorylated ERK (p-ERK) (E10; 1:200; Cell Signaling, Beverly, Mass.) were reacted at 4° C. for 12 hours. Thereafter, the resultant neurons were stained at RT for 1 hour with two fluorescent antibodies, that is, Alexa Fluor 568 goat anti-rabbit IgG and Alexa Fluor 488 goat anti-mouse IgG (1:200; Molecular Probes, Eugene, Oreg.). After staining, ERK-positive neurons among TH-positive neurons were examined.

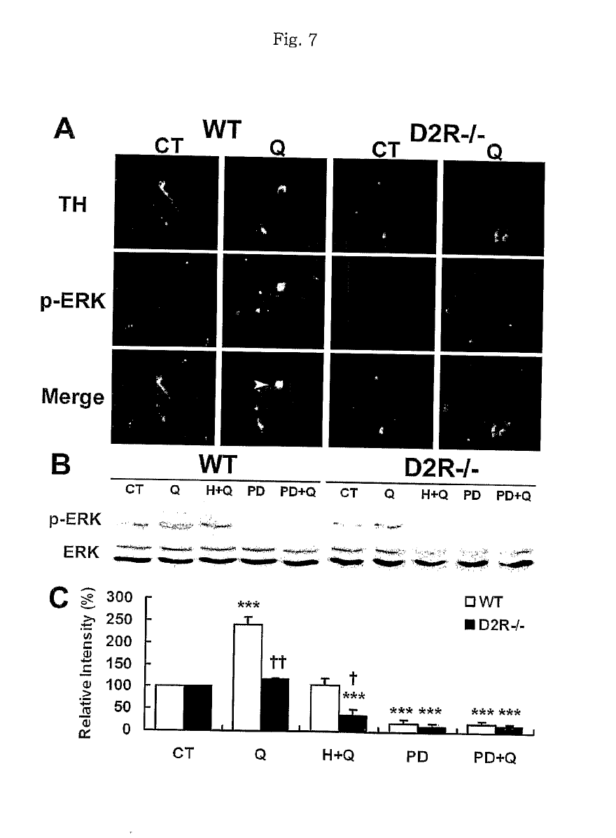

To observe immunofluorescent images, a confocal microscope system (Nikon Eclipse fluorescence microscope, TE2000-U, Nikon, Kanagawa, Japan; Ultraview RS confocal scanner, Perkin elmer, Wellesley, Mass.) or a fluorescence microscope (Axiovert 2000 microscope with epifluorescence unit, Zeiss, Zena, Germany) was used. The Western blot analysis was carried out in the following conditions. After treatment with 10 μM quinpirole for 15 minutes, followed by washing with ice-cold PBS and lysed in a buffer containing 20 mM Tris, pH 7.5, 150 mM NaCl, 1 mM EDTA, 1 mM EGTA, 2.5 mM sodium pyrophosphate, 1 mM glycerol phosphate, 1 mM Na₃VO₄, 1 μg/ml aprotinin, 1 μg/ml leupeptin, 1 mM PMSF, and 1% Triton X-100 for 10 minutes on ice. The resultant mixture was homogenized with probe type sonicator on ice, and followed by centrifugation at 13,000×g for 10 minutes at 4° C. Protein (50 μg), as measured by Bradford protein assay, was subjected to electrophoresis on 10% SDS-PAGE. The protein separated by electrophoresis was transferred to a polyvinylidene difluoride nitrocellulose membrane using a semi-dry transfer unit (Amersham bioscience, Piscataway, N.J.) and primary reaction was carried out on primary antibodies, that is, a mouse monoclonal anti-p-ERK (1:2000; Cell Signaling, Beverly, Mass.) and a rabbit monoclonal anti-ERK (1:5000; Santa Cruz Biotechnology, Santa Cruz, Calif.), at 4° C. for 12 hours, followed by detecting specific blots by enhanced chemiluminescence (Amersham Biosciences, Piscataway, N.J.). Here, quinpirole treatment was made in the presence or absence of 50 μM PD98059 for 30 minutes or 1 μM haloperidol for 5 minutes. As a result, it was confirmed that phosphorylation of ERK was detected in quinpirole-treated WT neurons while no phosphorylated-ERK was detected in D2R−/− neurons (FIG. 7A). In addition, to investigate whether treatment with haloperidol and quinpirole (H+Q) might result in D2R-induced ERK activation detectable, WT and D2R−/− neurons were treated with H+Q and analyzed by Western blot. By contrast, when treated with haloperidol and PD98059, was detected in neither WT nor D2R−/− neurons showed phosphorylated ERK (FIGS. 7B and 7C).

After primary mesencephalic cultures were treated with quinpirole for 6 hours, immunofluorescence of Nurr1 was confirmed by immunostaining (FIG. 8A). In detail, the experimental procedure was carried out in the same manner as described above, except that a mouse monoclonal anti-TH (1:1000; Diasorin, MN) and a rabbit polyclonal anti-Nurr1 (M-196; 1:200; Santa Cruz Biotechnology, Santa Cruz, Calif.) were reacted at 4° C. for 24 hours. As a result, while no quinpirole-induced Nurr1 activation was detected in D2R−/− neurons, quinpirole-induced Nurr1 activation was detected in WT neurons. In addition, with regard to quantitative analysis of a ratio of Nurr1 positive cells to TH-positive neurons, after quinpirole treatment, a noticeable increase was shown in WT neurons (FIG. 8B).

### INDUSTRIAL APPLICABILITY

As described above, according to the present invention, activation of Nurr1 and development of dopaminergic neurons can be modulated, Nurr1-related diseases can be treated. Further, the present invention can simply screen efficacy of test compounds or clinical pharmaceutical compositions having therapeutic effects on Nurr1 related diseases such as neuropsychiatric diseases.

1. (canceled)

2. A method for screening a modulator of a dopamine D2 receptor, the method comprising:
contacting a test compound with dopaminergic neurons having the dopamine D2 receptor and ERK(extracellular signal regulated kinase);
measuring an increased or decreased activation level of ERK in the dopaminergic neurons; and
determining the test compound as an agonist of the dopamine D2 receptor when the activation level of ERK is increased in the presence of the test compound compared with the activation level of ERK in the absence thereof; or
determining the test compound as an antagonist of the dopamine D2 receptor when the activation level of ERK is decreased in the presence of the test compound compared with the activation level of ERK in the absence thereof.

3. The method of claim 2, wherein activation of ERK is phosphorylation of ERK.

4. The method of claim 2, wherein the agonist of the dopamine D2 receptor is used for development of the dopaminergic neurons.
